(19) United States
(12) Patent Application Publication
(10) Pub. No.: US 2008/0317790 A1
(43) Pub. Date: Dec. 25, 2008

(54) Process for Treatment of Kappa Carrageenan

(75) Inventor: Jens Eskil Trudsoe, Roskilde (DK)

Correspondence Address: J M HUBER CORPORATION, 333 THORNALL STREET, PATENT DEPARTMENT, EDISON, NJ, 08837-2220, US

(73) Assignee: CP KELCO U.S., INC., Atlanta, GA (US)

(21) Appl. No.: 11/767,729

(22) Filed: Jun. 25, 2007

(51) Int. Cl. A23L 1/0532; A23L 1/05; A23L 1/31; A61K 8/02; A61K 8/04; A61K 9/48

(52) U.S. Cl. 424/401; 424/452; 426/574; 426/575; 426/89

(57) ABSTRACT

The present invention relates to a process for treating precipitated carrageenan, comprising the steps of (a) treating the precipitated carrageenan with an aqueous treatment solution containing an alkali or a salt, (b) washing the treated precipitated carrageenan in water, and (c) drying the washed precipitated carrageenan.

### BACKGROUND OF THE INVENTION

Production of carrageenan can be traced back to Ireland where plants of the red seaweed algae species of chondrus crispus were first harvested with rakes during low tide or by gathering seaweed that had washed ashore. After harvesting, the weeds were typically washed, sun-bleached, dried and boiled with milk to form a pudding. The weeds themselves were dubbed “Irish Moss” and after making it familiar to most of Europe, Nineteenth Century Irish immigrants carried it to the U.S. and Canada as well.

Today, this seaweed pudding is mostly confined to Ireland's cultural history, but carrageenan has become much more important because of its effectiveness as a functional food additive in forming gels in an aqueous system, which make it useful in a wide variety of applications, including beer (in which it has been used for over 150 years as a fining) to processed meat and food products like milk drinks and deserts; pharmaceutical preparations such as orally-administered gel caps; personal care products such as toothpaste and skin care care preparations; and household products such air-freshener gel and cleaning gels. The temperature at which carrageenan gels and melts is dependent on a number of factors that include especially the concentration of gelling cations such as potassium and calcium ions. Generally speaking, the higher the concentration of gelling cations the higher the gelling and melting temperature of the carrageenan. Such cations may come not only from the composition to which the carrageenan is added as a gelling agent, but also from the carrageenan itself.

Thus, carrageenans with relatively high gelling cation concentrations also require relatively high-temperature processing. Generally, lower temperature processes are preferred since these save processing time, are less expensive and don't negatively affect the preparation of the composition in which the carrageenan is being included—this is especially important for food compositions, where higher temperatures may impair the base foodstuffs that are included in the food product. Thus, in order to produce carrageenan materials that promote gelling at even lower temperatures there is a continuing need for carrageenan extraction methods that reduce the concentration of gelling cations in the carrageenan.

Contemporary methods of carrageenan extraction and production have advanced considerably in the last fifty years. Perhaps most significantly is that today; rather than being gathered from wild-grown seaweed, carrageenan-containing plants such as Kappaphycus cottonii (Kappaphycus alvarezii), Euchema spinosum (Euchema denticulatum), and the above mentioned Chondrus crispus are more commonly seeded along nylon ropes and harvested in massive aqua-culture farming operations particularly in parts of the Mediterranean and throughout much of the Indian Ocean and along the Asian Pacific Ocean Coastline. Just as in the Nineteenth-century process, in contemporary processes before further processing the seaweed raw materials are first thoroughly cleaned in water to remove impurities and then dried. Then, as described in U.S. Pat. No. 3,094,517 to Stanley et al. the carrageenan is extracted from the cleaned seaweed while also at the same time being subjected to alkali modification by placing the seaweed in solution made slightly alkaline by the addition of a low concentration of alkali salt (i.e., a pH of the solution is raised to a range of, e.g., 9-10) and then heating this solution to a temperature of around 80° C. for a period of time of about 20 minutes to as long as two hours.

Subjecting the carrageenan-containing seaweed to alkali modification has the desired result of reducing the gelling cation concentration in the resulting carrageenan product; however, the extent to which the gelling cation levels can be reduced is limited because only relatively low concentrations of alkali may be used so as to not depolymerise (and thus damage) the carrageenan in the seaweed. So even though the gelling cation concentrations are reduced, they still remain high.

For example, when an alkali modification process is NOT used, typical cation concentration levels in kappa carrageenan are:
Potassium: About 4%
Calcium: About 0,4%
Magnesium: About 0.5%
Sodium: About 2%

When an alkali modification step is used to reduce these gelling cation concentrations, such as in U.S. Pat. No. 3,094,517 (Stanley et al), which makes use of calcium hydroxide as alkali modification agent, the resulting cation concentration levels are:
Potassium: About 5%
Calcium: About 2%
Magnesium: About 0.01%
Sodium: About 1%

As can be seen, the alkali modification step taught in U.S. Pat. No. 3,094,517 significantly reduced the levels of magnesium and sodium ions, but not other gelling cations such as potassium and calcium.

Given the foregoing there is a need in the art for a process for reducing the concentration of gelling cations, arid thereby lowering the gelling and melting temperatures, without depolymerising the carrageenan or damaging it in some other way.

### BRIEF SUMMARY OF THE INVENTION

Disclosed in the present invention is a process for treating precipitated carrageenan, comprising the steps of (a) treating the precipitated carrageenan with an aqueous treatment solution containing an alkali or a salt, (b) washing the treated precipitated carrageenan in water, and (c) drying the washed precipitated carrageenan.

Also disclosed in the present invention are products made by this process, including food products, household cleaning products, and personal care products.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows the effect of alkali treatment time on the gelling and melting temperatures of gels made with neutral extracted kappa carrageenan.

FIG. 2 shows the cation composition of neutral extracted kappa carrageenan treated with alkali.

FIG. 3 shows the gelling and melting temperatures of gels made with Traditional Kappa carrageenan having been subject to different alkali treatment times.

FIG. 4 show's the cation content in Traditional Kappa carrageenan treated with alkali for different periods of time.

FIG. 5 shows a comparison of gelling and melting temperatures of alkali treated neutral extracted kappa carrageenan and traditional kappa carrageenan.

FIG. 6 shows salt treatment and gelling and melting temperatures of gels made with neutral extracted kappa carrageenan.

FIG. 7 shows the cation composition of salt treated neutral extracted kappa carrageenan.

FIG. 8 shows gelling and melting temperatures of salt treated traditional kappa carrageenan.

FIG. 9 shows the cation content in salt treated traditional kappa carrageenan.

FIG. 10 shows a comparison of gelling and melting temperatures of salt treated neutral extracted and traditional kappa carrageenan.

FIG. 11 shows gelling and melting temperatures during alkali treatment of wet precipitated neutral extracted kappa carrageenan.

FIG. 12 shows the cation composition of neutral extracted wet precipitate of kappa carrageenan.

FIG. 13 shows gelling and melting temperatures of alkali treated wet precipitate of tradition kappa carrageenan.

FIG. 14 shows the cation content in alkali treated wet precipitate of traditional kappa carrageenan

FIG. 15 shows a comparison of gelling and melting temperatures of alkali treated wet precipitate of neutral extracted and traditional kappa carrageenan.

FIG. 16 shows a comparison of break strength of alkali treated wet precipitate of neutral extracted and traditional kappa carrageenan.

FIG. 17 shows gelling and melting temperatures of salt treated wet precipitate of neutral extracted kappa carrageenan.

FIG. 18 shows the cation composition of salt treated wet precipitate of neutral extracted kappa carrageenan.

FIG. 19 shows gelling and melting temperatures of salt treated wet precipitate of traditional kappa carrageenan.

FIG. 20 shows the cation composition of salt treated wet precipitate of traditional kappa carrageenan,

FIG. 21 shows a comparison of gelling and melting temperatures of salt treated wet precipitate of neutral extracted and traditional kappa carrageenan.

FIG. 22 shows a comparison of break strength of salt treated wet precipitate of neutral extracted and traditional kappa carrageenan.

FIG. 23 shows a comparison of gelling and melting temperatures of alkali treated dry and wet precipitate of neutral extracted iota carrageenan.

FIG. 24 shows a comparison of gelling and melting temperatures of salt treated dry and wet precipitate of neutral extracted kappa carrageenan.

FIG. 25 shows a comparison of gelling and melting temperatures of alkali treated dry and wet precipitate of traditional kappa carrageenan.

FIG. 26 shows a comparison of gelling and melting temperatures of salt treated dry and wet precipitate of traditional kappa carrageenan.

FIG. 27 shows the effect of lower alcohol concentration during salt treatment on gelling and melting temperatures.

FIG. 28 shows the cation composition of salt treated wet precipitate of traditional kappa carrageenan using low and high concentrations of ethanol during treatment.

FIG. 29 shows a temperature sweep graph.

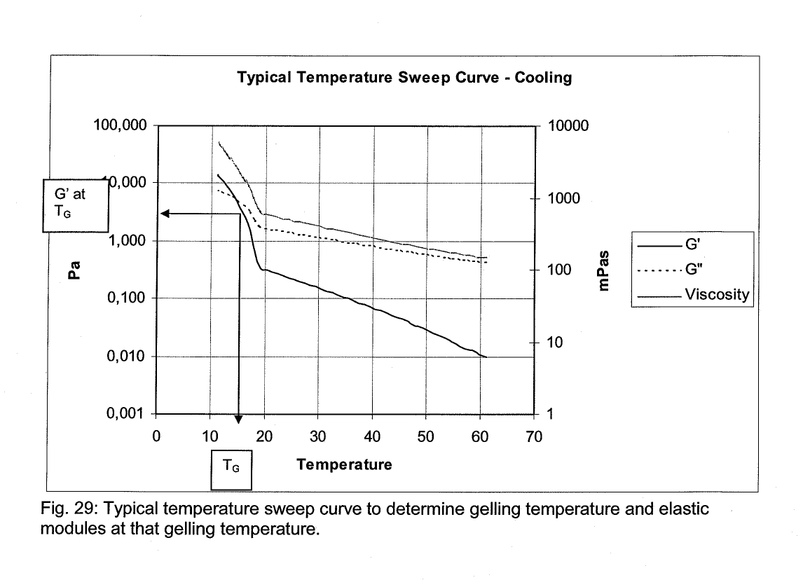

FIG. 30 shows a temperature sweep graph.

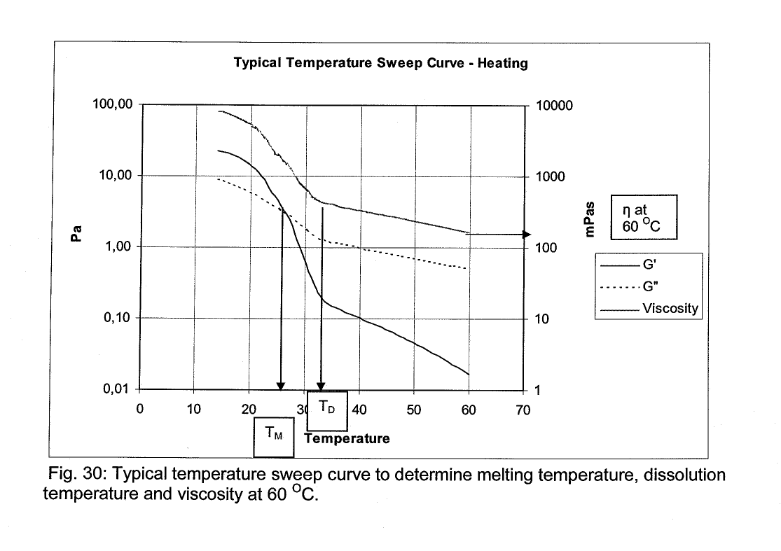

### DETAILED DESCRIPTION OF THE INVENTION

All parts, percentages and ratios used herein are expressed by weight unless otherwise specified. All documents cited herein are incorporated by reference.

By “alkali” it is meant a base according to the Brønsted-Lowry definition, i.e., an alkali is a molecule or ion that accepts a proton in a proton-transfer reaction.

The present invention is directed to kappa carrageenans, which may be more specifically described as generic repeating galactose and 3,6-anhydrogalactose residues linked b-(1-4) and a-(1-3), respectively and with characteristic 4-linked 3,6-anhydro-a-D-galactose and 3-linked-b-D-galactose-4-sulphate groups—kappa carrageenans differ from iota carrageenans only by the presence of a single sulphate group. The molecules arrange themselves in a right-handed double helix with the strands parallel and threefold, again iota and kappa carrageenan are very similar in this regard, with kappa carrageenan forming a slightly more disordered helix. The helix is stabilized by interchain hydrogen bonds through the only unsubstituted positions at O-2 and O-6 with the sulphate groups projecting outward from the helix. As mentioned above, there is a strong correlation between the presence of gelling cations and gellation. Without being limited by theory, it is believed that gels are formed in kappa carrageenan through gelling (primarily monovalent) cations such as Na, K, Rb, Cs, NH₄, Ca²⁺ as well as some divalent cations like calcium atoms that facilitate side-by-side interaction of the strands to form a three dimensional gel network. The exact transformation mechanism from the carrageenan as randomly-oriented coils at higher temperatures to a gelled network is the subject of some dispute. As the temperature is lowered the random coils of carrageenan molecules reaggregate to form gels. In one model of gellation, a gel is created by the formation of the carrageenan molecules into double helices; in certain forms of carrageenan (such as kappa carrageenan) these double helices may themselves aggregate side-by-side due to the influence of the aforementioned gelling cations forming aggregates of double helices and eventually even forming domains of a three-dimensional ordered gel network. Alternatively it has been suggested that upon cooling the random coils of the carrageenan molecules do not form double helices but only single helix structures, and that these single helix structures form single helices in which the gelling cations nested in the bends of the helix promote intermolecular aggregation.

Accordingly, the present invention is directed towards a process for producing kappa carrageenan with substantially reduced levels of gelling cations. Particularly, the present invention relates to treatment of precipitated seaweed extracts with salt or alkali compounds. Of equal importance is that this treatment process reduces the gelling cation concentration without extracting the carrageenan; in other words, depleting the gelling cations of the carrageenan by performing the alkali modification process essentially in situ. By modifying the polymer in situ in the seaweed, depolymerisaton of the carrageenan polymer is avoided and a kappa carrageenan preparation is produced that forms gels having lower gelling and melting temperatures than were hitherto known. The present invention relates to the surprising discovery that through various treatments with salts or alkali of either wet or dried precipitated seaweed extracts that the polymer situated in the seaweed precipitate can be modified in situ to provide a preparation, which forms gels having controlled gelling and melting temperatures.

As mentioned above, unlike other carrageenan refining processes, the present one begins not with seaweed raw material but instead seaweed extract precipitate. Methods for preparing precipitate are well-known to those of ordinary skill in the art. One of the most common of such methods is described in U.S. Pat. No. 3,094,517 read in combination with U.S. Pat. No. 3,907,770, in which seaweed is extracted at high temperatures with a surplus of calcium hydroxide and then left for an extended period of time at high pH to accomplish complete alkali modification of the polymer. Another suitable technique is disclosed in U.S. Pat. No. 5,801,240 where potassium hydroxide-treated seaweed, after treatment and wash, can be extracted at high temperature with water. Yet another method is disclosed in U.S. Pat. No. 5,502,179, where potassium chloride is used to form the carrageenan precipitate.

The process for producing carrageenans according to the present invention will now be described in greater detail.

The precipitate is obtained using one of the aforementioned processes or some other suitable process. The precipitate may be dried and optionally milled, or alternatively may be pressed, wet precipitate.

After obtaining the precipitate, the precipitate is treated with an aqueous treatment solution containing at least one of alkali or salt in water. The alkali and salt provide cations, which exclude potassium, calcium and/or magnesium in the carrageenan, while the concentration of the alkali in the treatment solution is held sufficiently high to reduce the aqueous solubility of the carrageenan thus preventing it from leaching out of the seaweed and dissolving into the water during this and subsequent steps.

Accordingly, by treating the carrageenan-containing seaweed in this way, the carrageenan is depleted from its gelling cations in situ.

Preferred alkalis are sodium hydroxide and its corresponding carbonates and bicarbonates, with sodium hydroxide being the most preferred. Sodium hydroxide is particularly notable for reducing the gelling and melting temperatures of carrageenan. Also suitable is calcium hydroxide. As discussed above, the concentration of the alkali must be such to provide sufficient cations while preventing solubilization of the carrageen in the water phase; an appropriate range to accomplish this dual purpose is a concentration of alkali in range of 3-30 wt %, preferably 10-25 wt % and most preferably 15-20 wt %.

In some cases alcohol may be added to the treatment solution to further reduce the leaching out of the carrageenan from the seaweed and its dissolving into water. It is particularly important to add alcohol when relatively small quantities of the aqueous treatment liquid are used. This is because excess water initially present in the wet seaweed and also remaining from the washing step could dilute the concentration of the cations in the aqueous treatment solution to the point that the carrageenan begins to leach out. The presence of alcohol in the treatment solution helps maintain high yields, especially as the treatment temperature is increased. Preferred alcohols are methanol, ethanol and isopropyl alcohol with ethanol being most preferred. The amount of alcohol ranges from 200-800 ml alcohol per 1000 ml treatment solution, preferably 200-600 ml alcohol per 1000 ml treatment solution and most preferably 200-500 ml alcohol per 1000 ml treatment solution.

The temperature during treatment ranges from 0-70° C., preferably 5-50° C. and most preferably 5-25° C. The treatment time is in the range of about 1 minute to about 24 hours, preferably about 1 minute to about 5 hours, and most preferably about 1 minute to 80 minutes.

Either a batch wise or counter current process may be used; although the counter current process is preferred because it makes better utilisation of the treatment liquid.

Carrageenan products treated with alkali have gelling temperatures in the range of about 20° C. -37° C., preferably about 20° C.-31° C. and most preferably about 20 to about 22° C.; and melting temperatures in the range of about 38 to about 63° C., preferably about 38 to about 49° C. and most preferably about 38 to about 40° C. In addition, carrageenan products according to the first embodiment are characterized by a sodium content in the range 4.050-7.310%, preferably 4.420-7.310% and most preferably 5.440-7.310%; a potassium content of 0.320%-4.560%, preferably 0.320-0.910% and most preferably 0.320-0.640%; a calcium content of 0.300-1.990%, preferably 0.300-1.790% and most preferably 0.300-1.620%; and a magnesium content of 0.012-0.630%, preferably 0.012-0.600% and most preferably 0.012-0.580%.

Alkalis include sodium hydroxide, sodium carbonate and sodium bicarbonate. The preferred alkali is sodium hydroxide. The concentration of alkali in the water phase is 3-30%(w/w), preferably 10-25% (w/w) and most preferably 15-20% (w/w).

Carrageenan products treated with salt have gelling temperatures in the range 19-37° C., preferably 19-24° C. and most preferably 19-22° C.; and melting temperatures in the range 37-63° C., preferably 37-42° C. and most preferably 37-40° C. In addition, carrageenan products according to the second embodiment are characterized by a sodium content in the range 3,730-6,990%, preferably 4,190-6,990% and most preferably 4,310-6,990%; a potassium content of 0,840-4.560%, preferably 0,840-1,730% and most preferably 0,840-1,490%; a calcium content of 0,080-1.750%, preferably 0,080-0,500% and most preferably 0,080-0,420%; and a magnesium content of 0,005-0,610%, preferably 0,005-0,030% and most preferably 0,005-0,023%.

Salts include sodium salts like sodium chloride, sodium sulphate, sodium phosphate, sodium tripolyphosphate and sodium hexanietaphosphate. The concentration of sodium salt in the water phase is in the range 3-30 wt %, preferably 10-25 wt %, and more preferably 15-20 wt.%.

In the third step in the process the treated seaweed is subjected to washing to remove the excess of the last reagent that was used in the second or treatment step. The reagent can of course be either a salt or an alkali. Washing is done with slow agitation and the number of washings is in the range 1-4, preferably 1-2, and washing time is in the range 10-30 minutes per wash, preferably 15 minutes per wash. Controlling the number of washing steps is important because the yield decreases with time (possible reasons for this are discussed below) and because the number of washing steps affects the gelling and melting temperatures (again, this is discussed in greater detail, below). As above to limit leaching out of the carrageenan from the seaweed the temperature during washing is held in the range 0-25° C., preferably 0-5° C.

In the fourth and final step of the process the treated seaweed can be dried and ground into a carrageenan powder.

Other aspects of the processes for production of carrageenan according to the present invention are not particularly limited, and where necessary conventional carrageenan technology may be used. In addition to the specific steps set forth herein, processes of the present invention may further comprise additional processes typically associated with carrageenan production.

In this area, where gelling and/or melting must take place at lower temperatures than what is possible with conventional carrageenan products, applications include but are not limited to:

Air freshener gels: these gels contain one or more non-ionic surfactants, and when the gels are heated above a certain point (referred to as the “cloud point”, typically non-ionic surfactants have a cloud point in the range of about 0 to about 60° C.) the non-ionic surfactants become less soluble and precipitate out of the gel leading to a cloudy, non-transparent gel. Typically, conventional carrageenan products display gelling temperatures above the cloud point of the surfactants, and thus, freeze the surfactant crystals in the gel, causing the gel to become permanently unclear even when the temperature is lowered below the cloud point. The carrageenan products of the present invention can be tailored to gel at or below the cloud point of the surfactant, thus, preventing the surfactant crystals from being froze in the gel and so preventing the resulting air freshener gel from becoming cloudy, and non-transparent.

Cold setting air freshener gels: Conventional air freshener gels are made by heating the composition to about 70-90° C., after which gelation takes place during cooling. However, the heating provides for a substantial loss of the fragrance used in the air freshener formulation as some of the fragrance material evaporates during heating. Carrageenan products of the present invention can be tailored to dissolve at temperatures at or below room temperature, which eliminates the loss of fragrances. Once dissolved, the liquid air freshener formulation can be poured into its final container, which contains gelling cations (as discussed above) that in conjunction with the carrageenan form the gel network. Such cations may be added directly into the container before filling the air freshener formulation into the container, or the cations may be added as a coating, such as a film coating, with which the container is pre-coated. As the cations diffuse into the air freshener formulation under quiescent conditions, the air freshener formulation will gel into a homogeneous gel.

Water-in-oil emulsions; Water-in-oil emulsions are characterized by a continuous oil phase in which a discontinuous phase of water droplets are dispersed. In many cases it is desired that the water-in-oil emulsion inverts into an oil-in-water emulsion at a specific temperature so that the emulsion releases its water soluble constituents. An example is margarine, where the emulsion inverts in the mouth to release water soluble aromas and salts. Gelatine is the preferred stabilizer of the water phase, since gelatine ensures that the aqueous phase melts at the same temperature as the oil phase. That temperature is about the temperature in the mouth, and thus, through the saliva and the shear in the mouth, the emulsion inverts to an oil-in-water emulsion and releases aroma and salt. Conventional carrageenan products are unable to form gels, which melt at the temperature in the mouth, but carrageenan products of the present invention can be tailored to do just that.

Similarly, most skin care lotions are produced as oil-in-water emulsions. This means that the water phase is the continuous phase, which requires that preservatives are used in skin care lotion formulations. There is a desire to eliminate preservatives in skin care lotions, particularly preservatives of the parabene type, because they have some similarity with hormones. Carrageenan products of the present invention makes it possible to provide a skin care lotion in the form of an water-in-oil emulsion, which because of the oil continuous phase does not require preservatives, but which will invert to a spreadable oil-in-water emulsion at the temperature of the skin and the shear from rubbing in the lotion.

Capsules: Soft capsules are made trough sealing of two capsule halves. Gelatine is preferred because gelatine forms capsules which can sealed at low temperatures through the low melting temperature of gelatine gels. There is, however, a desire for an alternative to gelatine that meets the dietary guidelines of vegetarians, Jewish kosher, and halal practitioners, and is not derived from meat products association with Bovine Spongiform Encephalopathy. Prior art carrageenan products could not be used in this application because they form gels with much higher melting temperatures. But Carrageenan products of the present invention can be tailored to form gels, which melt at the same or even lower temperatures than gelatine gels.

Encapsulation; Encapsulation is used in areas such as flavour encapsulation and encapsulation of drugs. In cases where the agent being encapsulated are heat sensitive, carrageenan products of the present invention can encapsulate the agent at low temperatures. Similarly, the encapsulated ingredient can be released at any temperature in the range from below 0° C. and up to about 75° C., preferably about about 30° C. to about 40° C. depending on the composition of the encapsulating formulation.

Processed meat, poultry and fish products: Processed meat, poultry and fish products are often heat treated at pasteurization temperature, which is about 72° C. The aqueous phase of such products typically contain up to about 3% sodium chloride, which precludes the dissolution of conventional carrageenan products. Carrageenan products of the present invention can be tailored to dissolve at a temperature at or below the pasteurization temperature, which leads to dissolution of the carrageenan product and thus, a more homogeneous gel in the final processed meat, poultry or fish product.

Dentifrice and Toothpaste Products: in these carrageenan products of the present invention provide for higher viscosity due to their increased solubility. This increased solubility of the carrageenan means there is more reactive carrageenan to form a viscous paste with the other ingredients in the dentifrice or toothpaste formulation—particularly the humectant and salts.

The present invention will now be explained in greater details with respect to the following several experiments. These experiments and their accompanying textual descriptions, will present detailed descriptions of the process of the present invention as well as results obtained from the experimental process. Additionally analysis of the results will be presented and supplemented by possible theoretical explanations. The following experimental equipment, materials and methods were used in carrying out the present experiments. Application of these experimental methods are introduced in the specific examples section below that illustrate the present invention and place it within the context of the prior art.

Equipment
Hobart mixer equipped with heating and cooling jacket and stirrer Hobart N-50G produced by Hobart Corporation, USA.
Cooling unit capable of cooling to about 5° C., e.g., the Haake K10/Haake DC10 produced by Thermo Electron GmbH, Germany.
Magnetic stirrer and heater equipped with temperature control, e.g., Ikamag Ret produced by Janke & Kunkel GmbH, Germany.
Beakers, 1 litre and 2 liters.
2 liters conical flask, Büchner funnel and vacuum pump.
Filter cloth.
Rheometer—Haake RheoStress RS100 equipped with cup Z20/48 mm and rotor Z20 DIN produced by Thermo Electron GmbH, Germany.
pH-meter—PHM220 produced by Radiometer, Denmark
Analytical balance, weighing with two decimals—Sartorius Basic B3100P produced by Sartorius GmbH, Germany.
Texture analyzer TA-TX2 equipped with 2 kg. weighing cell and 0.5 inch plunger traveling with a speed of 1 mm per second into the gel.
Crystallizing dishes having diameter of about 50 mm and height of about 20 mm.

Chemicals:
Sodium chloride, analytical, Merck KGaA, Darmstadt, Germany
Sodium hydroxide, analytical, Merck, Germany
Calcium hydroxide, analytical, Merck
Sodium methyl-4-hydroxybenzoate, analytical, Merck
Potassium chloride, analytical, Merck
Ethanol, 96%
Isopropyl alcohol, 100%
Glycerine, analytical, Schariau Chemie, Barcelona, Spain
Lemon oil, H. N. Fusgaard, Roedovre, Denmark
Cremophor RH 40, BASF, Ludwigshafen, Germany

Extraction of seaweed with demineralized water:
1. Seaweed was washed three times in 1 liter dematerialized water and kept in refrigerator.
2. About 130 g washed seaweed was placed in a 10-liter beaker.
3. 7500 ml boiling demineralized water was added and extraction performed at 90° C. for 1 hour.
4. The extracted seaweed was filtered using diatomaceous earth as filter aid.
5. The filtered extract was precipitated in three volumes 100% isopropanol, pressed by hand and dried at 70° C. over night.
6. In one embodiment, the pressed precipitated was treated without drying.

Treatment of seaweed extract:
1. The dried and milled precipitate was placed in a Hobart mixer.
2. Treatment agent was dissolved in demineralized water and ethanol.
3. The precipitate was treated with this mixture at 25° C. for various periods of time.
4. After treatment, the treated precipitated was washed twice at 5° C. with a mixture of demineralized water ethanol.
5. The washed precipitated was isolated and dried at 70° C. over night and milled on 0,250 mm screen.

The Determination of gelling and melting temperatures of carrageenan-compositions was made using a composition with the following carrageen-incorporating composition:

| Ingredients | Grams | % |
|---|---|---|
| Seaweed extract | 0.48 | 0.96 |
| Glycerine | 3.00 | 6.00 |
| Parabene | 0.05 | 0.10 |
| Demineralized | 33.75 | 67.50 |
| Water | | |
| Lemon oil | 1.25 | 2.50 |
| Isopropyl alcohol | 1.50 | 3.00 |
| Cremophor RH 40 | 10.00 | 20.00 |
| Net weight | 50.00 | 100.00 |

This composition was prepared as follows:
1. The water, glycerine and parabene were mixed.
2. The seaweed extract was dispersed in this mixture and stirred for about 60 minutes.
3. The dispersion was heated while stirring to 70° C.
4. The dispersion was then cooled to 55-60° C.
5. A hot (about 50° C.) preparation of lemon oil, isopropyl alcohol and Cremophor RH 40 was mixed into the cooled dispersion.
6. The net weight was adjusted with hot (about 60° C.) water and cooled over night at room temperature.

The gelling and melting temperatures were measured by temperature sweeps on Haake RheoStress RS100, using cooling and heating rates of 1° C./min. The following program was generally used, however, in some instances where gelling and melting temperatures were higher; the program was run at higher starting temperatures and lower end-temperatures:
1. 65-5° C., 0,50 Pa, f=0,4640 Hz
2. 5-65° C., 0,50 Pa, f=0,4640 Hz
3. Gelling temperature is defined as the temperature during the cooling sweep, where the elastic modulus, G′ intersects with the viscous modulus, G″.
4. Melting temperature is defined as the temperature during the heating sweep, where the elastic modulus, G′ intersects with the viscous modulus, G″.

FIG. A and FIG. B show typical temperature sweep graphs. The determination of break strength and gel strength of carrageenan-compositions was made using a composition with the following carrageen-incorporating air-freshener composition:

| Ingredients | Grams | % |
|---|---|---|
| Seaweed extract | 0.58 | 0.96 |
| Glycerin | 3.60 | 6.00 |
| Parabene | 0.06 | 0.10 |
| Water | 40.44 | 67.40 |
| KCl | 0.12 | 0.20 |
| Lemon oil | 1.50 | 2.50 |
| IPA | 1.80 | 3.00 |
| Cremophor | 12.00 | 20.00 |
| Net weight | 60.00 | 100.16 |

1. Water, glycerin, potassium chloride and parabene were mixed.
2. Seaweed extract was dispersed in this mix and stirred for about 60 minutes.
3. The dispersion was heated while stirring to 70° C.
4. The solution was cooled to 55-60° C.
5. A hot (about 50° C.) preparation of lemon oil, isopropyl alcohol and Cremophor RH 40 was mixed into the cooled solution.
6. The net weight was adjusted with hot (about 60° C.) water and cooled over night at room temperature.
7. The compression curved was established on Texture Analyzer T.A-TX2 using the following parameters:
   Plunger: 0,5 inch in diameter
   Plunger speed: 1 mm/sec
   Maximum penetration: 10 mm
   Gel strength measured at 2 mm compression

### EXAMPLES

The invention will now be described in more detail with respect to the following non-limiting examples which were performed with the above described equipment, materials and methods.

The following Examples relate to results obtained by treating the red seaweed Eucheuma cottonii with various treatment, compounds. The results obtained from the present invention were compared with comparative, prior art neutral extractions, in which the washed seaweed was extracted in demineralized water for one hour at 90° C.

T_{G} and T_{M} stand for gelling temperature, and melting temperature, respectively, while T_{D} is the dissolution temperature, and η stands for intrinsic viscosity at 60° C. The “% yield” is calculated as: % yield=(g. dry precipitate×1500×100)/(g. seaweed×g. precipitated extract×seaweed dry matter). Since yield of polymer from seaweed changes with season and with seaweed harvesting location, the yield of neutral extractions of seaweed have been assigned an index of 100, and subsequent calculations of yield index utilize that baseline figure.

Several experiments were performed with compositions prepared according to the present invention. The first step of the preparation of these compositions is extraction of carrageenan material from Eucheuma cottonii. Extracts were prepared both in a neutral extraction (marked “neutral extraction”, below) and in an alkali extraction conducted according to U.S. Pat. No. 3,094,517 and U.S. Pat. No. 3,907,770 (marked “traditional kappa”, below). In this “traditional kappa” method seaweed was extracted with a surplus of calcium hydroxide and left at high temperature for 24 hours to provide complete alkali modification. The extract was then filtered, neutralized to pH about 9 with carbon dioxide, filtered again and precipitated in three volumes of 100% isopropanol. After pressing, the precipitate was dried at 70° C. over night. The results are set forth in Tables 1 and 2, below.

TABLE 1

| | | Dry | Amount | | | T_{G} | T_{M} | T_{D} | Na | K | Ca | Mg | η | pH |
|---|---|---|---|---|---|---|---|---|---|---|---|---|---|---|
| Extract | Seaweed g | Matter % | Precipitated g | Precipitate g | Yield % | ° C. | ° C. | ° C. | Mg/g | Mg/g | Mg/g | Mg/g | cP | 1% |
| Neutral | 131.8 | 24.41 | 4195 | 14.73 | 65.48 | 33 | 56 | 55 | 14.10 | 38.40 | 3.80 | 6.10 | 185 | 8.15 |

TABLE 2

| | T_{G} | T_{M} | T_{D} | Na | K | Ca | Mg | η | pH |
|---|---|---|---|---|---|---|---|---|---|
| Extract | ° C. | ° C. | ° C. | Mg/g | Mg/g | Mg/g | Mg/g | cP | 1% |
| Tradi- | 37 | 63 | 65 | 4.00 | 45.60 | 17.50 | 0.14 | 778 | 9.09 |
| tional | | | | | | | | | |
| Kappa | | | | | | | | | |

(The dry matter of the seaweed was determined by drying the washed seaweed at 105° C. for 17 hours. T_{G} and T_{M} stand for gelling temperature and melting temperature, respectively, T_{D} is the dissolution temperature, and η stands for complex viscosity at 60° C. % yield is calculated as: % yield=(g. dry precipitate×7500×100)/(g. seaweed×g. precipitated extract×seaweed dry matter.)

As can be seen from the results in tables 1 and 2, traditional kappa carrageenan differs from neutrally extracted E. cottonii in that traditional kappa carrageenan; (1) provides gels with higher gelling and melting temperatures; requires higher temperatures for dissolution; (3) has a lower content of sodium and magnesium ions; and has a higher content of potassium and calcium ions.

The techniques of treating extracted carrageenan as taught in the present invention were then applied to these carrageenan extracts as set forth in the following detailed examples.

Treatment of Dry Precipitate with Alkali: The two extract preparations set forth above were treated with alkali. 16 g NaOH was dissolved in 80 ml demineralized water, and 120 ml 96% ethanol was added. The mixture was cooled to 25° C. About 2 g of extract was added, and the mixture stirred at 25° C. for various periods of time. After this treatment, the extract was isolated and washed twice at 5° C. with a mixture of 80 ml demineralized water and 120 ml 96% ethanol. The washed extract was isolated and dried over night at 70° C. and milled on a 0,250 mm screen. The results are set forth below in table 3 (which shows the treatment of neutral extract with alkali) and table 4 (which shows the treatment of traditional kappa with alkali):

TABLE 3

| | Treatment | | Extract | Extract | | | | | | | | | | |
|---|---|---|---|---|---|---|---|---|---|---|---|---|---|---|
| Extrac- | Temperature | Treatment | Before | After | | T_{G} | T_{M} | T_{D} | Na | K | Ca | Mg | η | pH |
| tion | ° C. | Time h | Treatment g | Treatment g | Yield % | ° C. | ° C. | ° C. | Mg/g | Mg/g | Mg/g | Mg/g | cP | 1% |
| Neutral | 25 | 0 | | | | 33 | 56 | 55 | 14.10 | 38.40 | 3.80 | 6.10 | 185 | 8.15 |
| | 25 | 1 | 2.14 | 2.02 | 94.39 | 21 | 41 | 43 | 54.40 | 3.70 | 3.90 | 6.30 | 167 | 10.81 |
| | 25 | 3 | 2.31 | 2.16 | 93.51 | 21 | 42 | 43 | 53.80 | 3.40 | 3.70 | 6.30 | 163 | 10.53 |
| | 25 | 5 | 2.15 | 2.00 | 93.02 | 22 | 42 | 43 | 50.30 | 3.20 | 3.70 | 6.30 | 175 | 10.54 |

TABLE 4

| | Treatment | | Extract | Extract | | | | | | | | | | |
|---|---|---|---|---|---|---|---|---|---|---|---|---|---|---|
| Extrac- | Temperature | Treatment | Before | After | | T_{G} | T_{M} | T_{D} | Na | K | Ca | Mg | η | pH |
| tion | ° C. | Time h | Treatment g | Treatment g | Yield % | ° C. | ° C. | ° C. | Mg/g | Mg/g | Mg/g | Mg/g | cP | 1% |
| Tradi- | 25 | 0 | | | | 37 | 63 | 65 | 4.00 | 45.60 | 17.50 | 0.14 | 778 | 9.09 |
| tional | 25 | 1 | 2.42 | 2.58 | 106.61 | 31 | 49 | 52 | 52.80 | 26.00 | 16.20 | 0.12 | 52 | 11.93 |
| Kappa | 25 | 3 | 2.17 | 2.40 | 110.60 | 27 | 45 | 46 | 73.10 | 17.80 | 16.70 | 0.13 | 42 | 11.96 |
| | 25 | 5 | 2.68 | 2.73 | 101.87 | 23 | 40 | 42 | 64.60 | 9.10 | 17.90 | 0.13 | 50 | 11.84 |

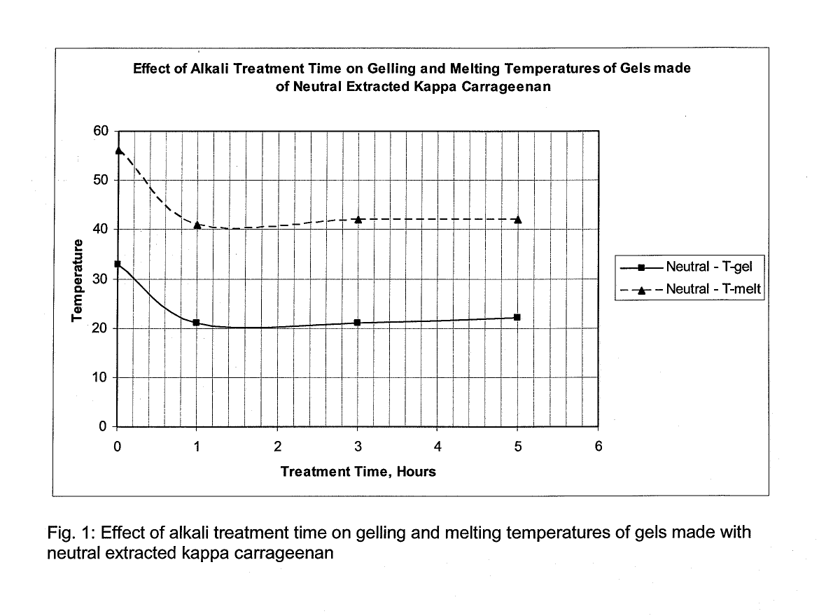

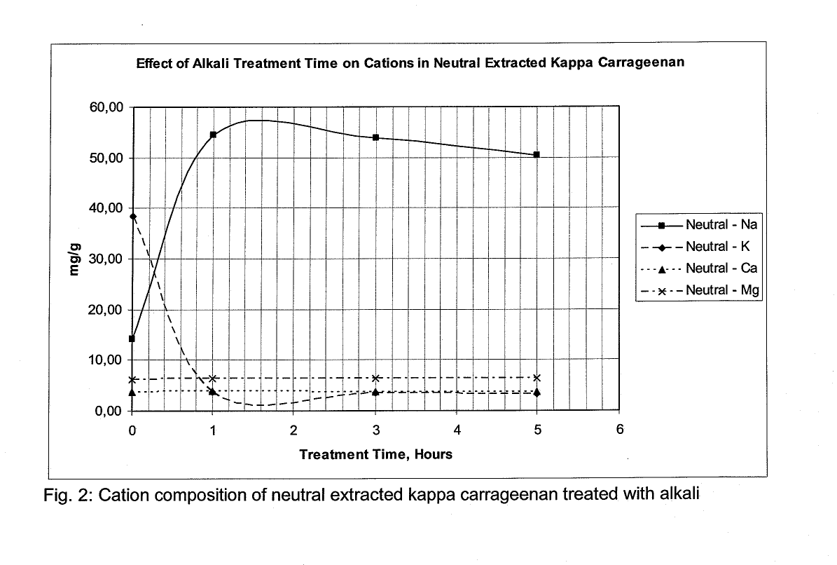

The results of table 3 are shown in FIG. 1 and FIG. 2. FIG. 1 shows that the gelling and melting temperatures decrease within the first hour of treatment. Thus, the gelling and melting temperatures can be controlled through treatment times in the range 0-1 hour. The gelling temperature decreases from about 35° C. to about 20° C. and the melting temperature from about 55° C. to about 40° C. FIG. 2 shows that during the first hours of treatment, the sodium content, in neutral extracted kappa carrageenan increases from about 1.5% to about 5.5%. The potassium content, however, decreases in the same period from about 4% to about 0.4%, whereas the content of calcium and magnesium stays constant at about 0.5% and 0.4%, respectively.

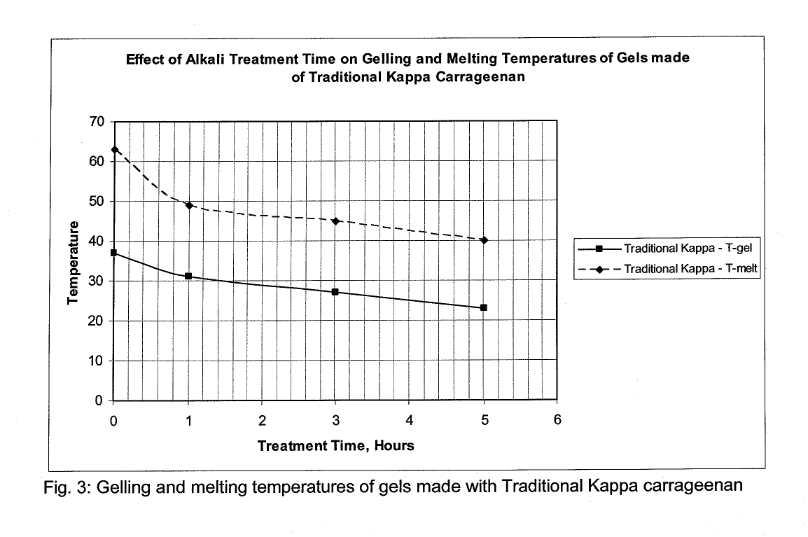

The results of table 4 are shown graphically in FIGS. 3, 4, and 5. As can be seen in the figures, the gelling and melting temperatures show the strongest drop during the first hour of treatment, but the gelling and melting temperatures continue to drop even after 5 hours of treatment. Thus, for traditional kappa carrageenan, the gelling and melting temperatures can be controlled over a wider range using longer treatment times. During five hours of treatment, gelling temperatures decrease from about 35° C. to about 20° C., and melting temperatures from about 65° C. to about 40° C.

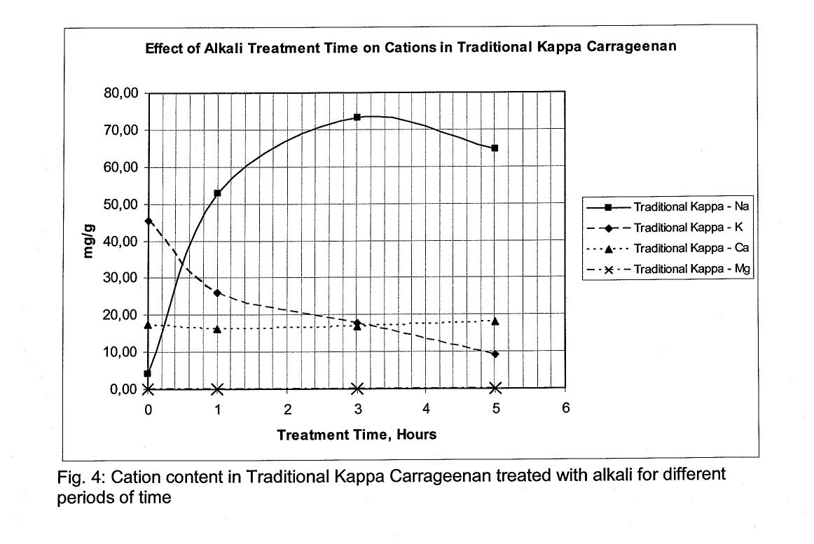

FIG. 4 shows that during the treatment with alkali, the sodium content in Traditional Kappa carrageenan increases from about 0.5% to about 7.5% during about three hours, after which a slight decrease occurs. However, the content of potassium continues to decrease even after five hours treatment. The potassium level goes from about 4.5% to less than 1%. Contrary, the levels of calcium and magnesium stay constant at about 1.7% and about 0.01%, respectively.

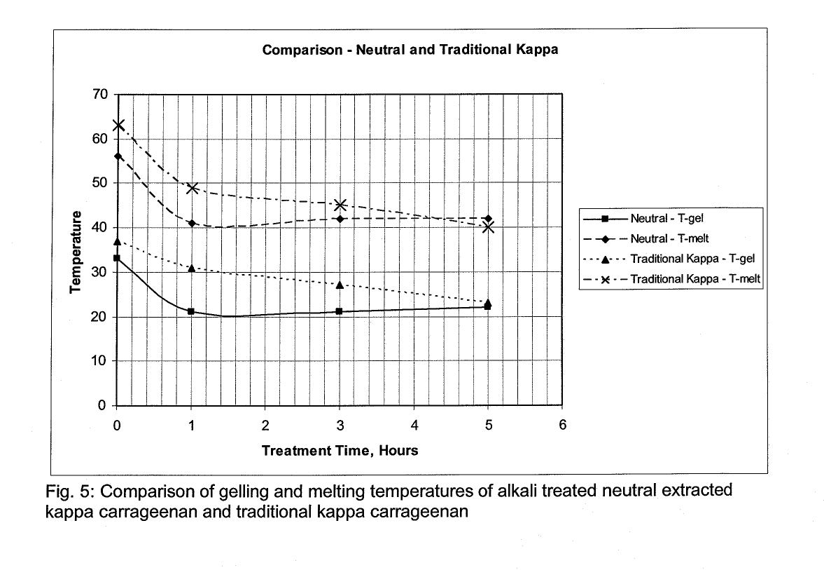

FIG. 5 shows the gelling and melting temperatures of the two preparations. In both cases, gelling and melting temperatures are reduced by about 10-15° C. during treatment times of a least 1-2 hours. The neutral extraction provides the lowest temperatures, being about 20° C. in gelling temperature and about 40° C. in melting temperature. This happened within 1-2 hours. However, the traditional extract's gelling and melting temperatures are reduced to about 30° C. and 45° C., respectively, within 1-2 hours, but continue to fall as treatment time increases. Thus, after five hours treatment, the gelling and melting temperatures of the traditional extracts are on par with the neutral extract. Gelling and melting temperatures can be controlled in the ranges: Neutral extract: T-gel: 35-20° C.; T-melt: 55-40° C. Traditional extract: T-gel: 40-20° C.; T-melt: 65-40° C. Put differently, the gelling and melting temperatures of traditional kappa carrageenan extracts can by the process of the present invention be reduced by about 20-25° C.

Treatment of dry precipitate with mil The two extract preparations set forth above were then treated with salt, 16 g NaCl was dissolved in 80 ml demineralized water, and 120 ml 96% ethanol was added. The mixture was cooled to 25° C. About 2 g of extract was added, and the mixture stirred at 25° C. for various periods of time. After this treatment, the extract was isolated and washed twice at 5° C. with a mixture of 80 ml demineralized water and 120 ml 96% ethanol. The washed extract was isolated and dried over night at 70° C. and milled on a 0.250 mm screen. The results are set forth below in table 5 (which shows the treatment of neutral extract with salt) and table 6 (which shows the treatment of traditional kappa with salt).

TABLE 5

| | Treatment | | Extract | Extract | | | | | | | | | | |
|---|---|---|---|---|---|---|---|---|---|---|---|---|---|---|
| Extrac- | Temperature | Treatment | Before | After | | T_{G} | T_{M} | T_{D} | Na | K | Ca | Mg | η | pH |
| tion | ° C. | Time h | Treatment g | Treatment g | Yield % | ° C. | ° C. | ° C. | Mg/g | Mg/g | Mg/g | Mg/g | cP | 1% |
| Neutral | 25 | 0 | — | — | | 33 | 56 | 55 | 14.10 | 38.40 | 3.80 | 6.10 | 185 | 8.15 |
| | 25 | 1 | 2.22 | 2.08 | 93.69 | 20 | 37 | 40 | 51.20 | 8.40 | 1.00 | 0.30 | 300 | 9.00 |
| | 25 | 3 | 2.35 | 2.12 | 90.21 | 20 | 37 | 40 | 51.10 | 8.50 | 1.00 | 0.29 | 302 | 9.06 |
| | 25 | 5 | 2.23 | 2.02 | 90.58 | 19 | 37 | 38 | 51.50 | 8.50 | 1.10 | 0.30 | 220 | 8.93 |

TABLE 6

| | Treatment | | Extract | Extract | | | | | | | | | | |
|---|---|---|---|---|---|---|---|---|---|---|---|---|---|---|
| Extrac- | Temperature | Treatment | Before | After | | T_{G} | T_{M} | T_{D} | Na | K | Ca | Mg | η | pH |
| tion | ° C. | Time h | Treatment g | Treatment g | Yield % | ° C. | ° C. | ° C. | Mg/g | Mg/g | Mg/g | Mg/g | cP | 1% |
| Tradi- | — | 0 | — | — | | 37 | 63 | 65 | 4.00 | 45.60 | 17.50 | 0.14 | 778 | 9.09 |
| tional | 25 | 1 | 2.39 | 2.31 | 96.65 | 30 | 50 | 52 | 37.30 | 23.60 | 6.60 | 0.07 | 58 | 7.70 |
| Kappa | 25 | 3 | 2.37 | 2.36 | 99.58 | 26 | 45 | 47 | 42.30 | 13.20 | 2.40 | 0.05 | 153 | 8.00 |
| | 25 | 5 | 2.28 | 2.17 | 95.18 | 24 | 42 | 44 | 46.50 | 14.90 | 2.70 | 0.05 | 69 | 8.84 |

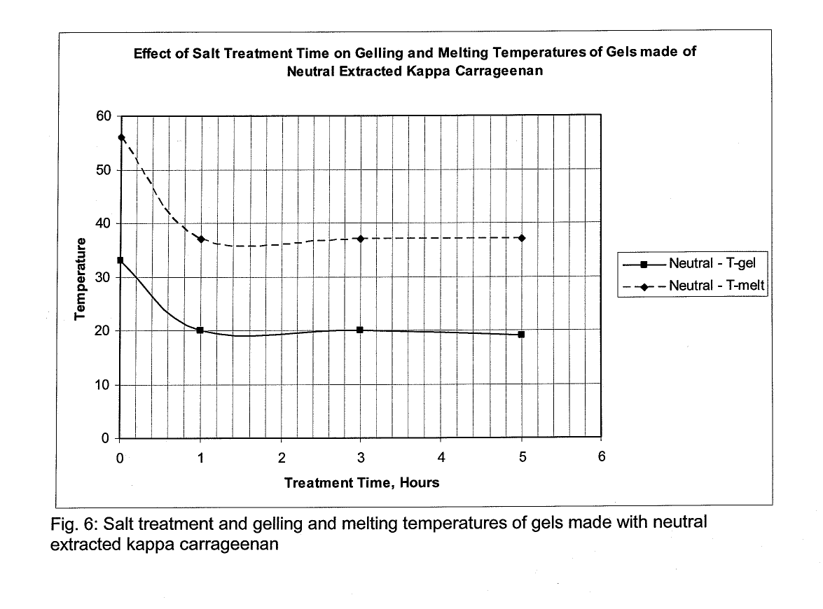

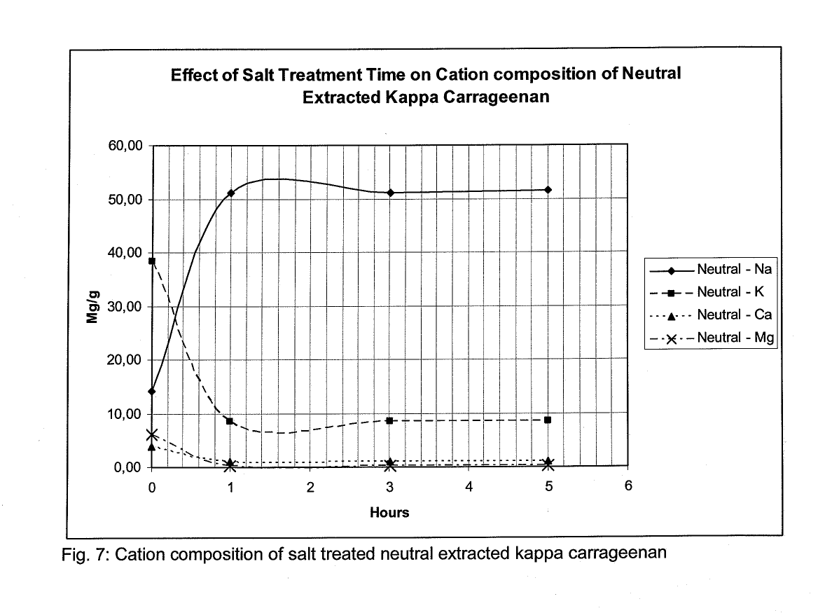

The results in table 5 are shown graphically in FIG. 6 and FIG. 7. FIG. 6 shows that gelling and melting temperature drop by about 20° C. within the first hour of treatment and then stays constant. Gelling temperature can be controlled within the range from about 35° C. to about 20° C., and melting temperature from about 55° C. to about 35° C.

FIG. 7 shows that within the first hour of salt treatment, the sodium content increases from about 1.4% to about 5.5%, whereas the potassium level decreases from about 4% to about 0.8%. The calcium level decreases from about 0.5% to about 0.1% and the magnesium level decreases from about 0.6% to about 0.03%.

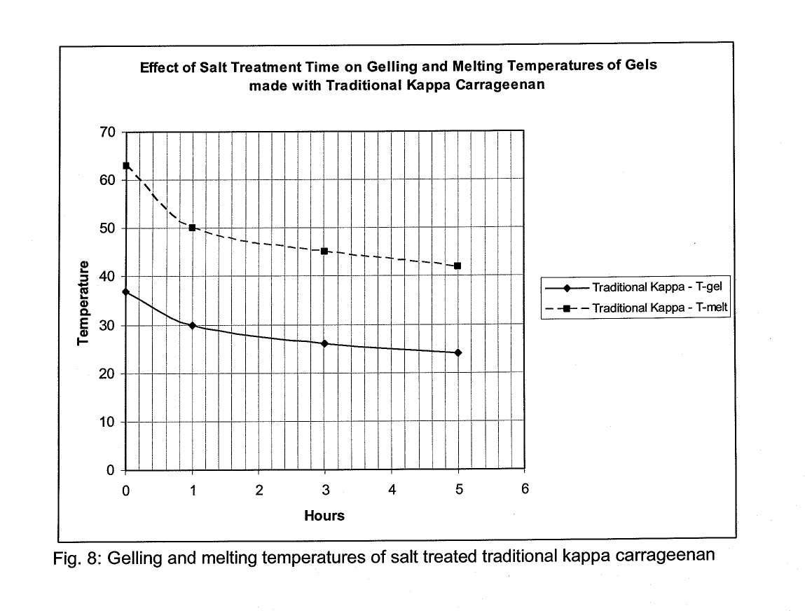

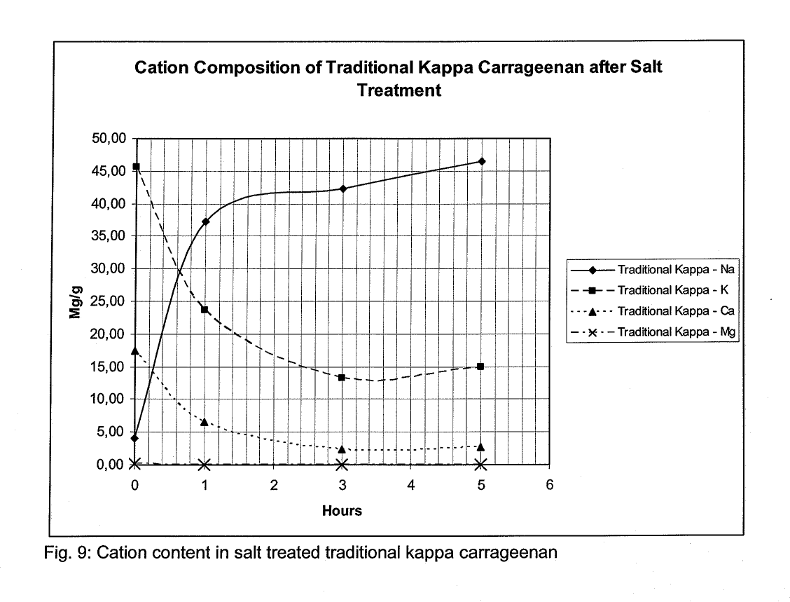

The results in table 6 are shown graphically in FIG. 8 and FIG. 9, FIG. 8 shows that during the first hour of treatment the gelling and melting temperatures of gels made with salt treated traditional kappa carrageenan continue to decrease during the entire period of salt treatment and beyond. Within five hours of treatment, the gel ling temperature decreases from about 37° C. to about 24° C. and the melting temperature decreases from about 63° C. to about 42° C.

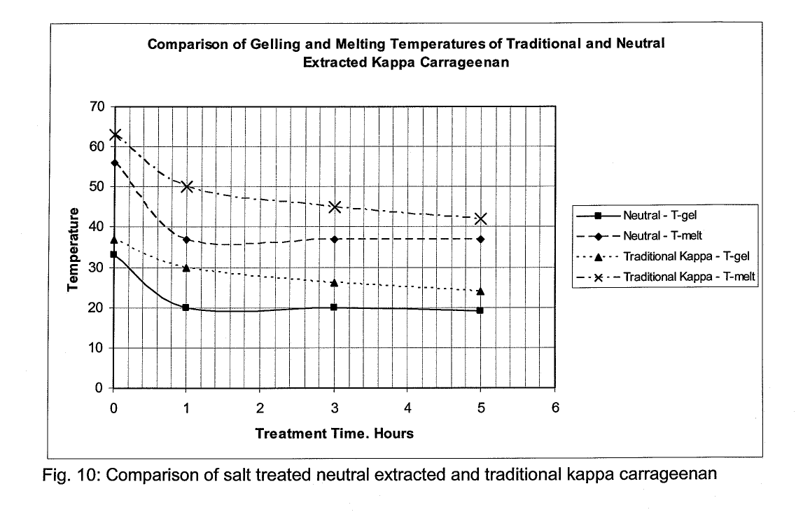

FIG. 9 shows that the sodium level continues to rise during the entire treatment period. During these five hours, the sodium content goes from about 0.4% to about 4.6%. The potassium level reaches a minimum after about three hours of treatment, and decreases from about 4.6% to about 1.3%. Also the calcium level, reaches a minimum after about three hours of treatment, and goes from about 1.8% to about 0.2%. Magnesium levels decrease from about 0.01% to about 0.005%. FIG. 10 compares the gelling and melting temperatures. The treated neutral extracted kappa carrageenan consistently provides gels of lower gelling and melting temperatures than gels made with traditional kappa carrageenan. The difference is of the order 5-10° C.

Several additional experiments were conducted with a new batch of carrageenan extract, from Eucheuma cottonii as above, except the material was not dried alter extraction—and so treatment was conducted on wet carrageenan. As above, batches of “traditional kappa” and neutral kappa were prepared. The results are set forth in Tables 7 (neutral extraction) and 8 (alkaline extraction), below.

TABLE 7

| Ex- | | | | | | | | | Break |
|---|---|---|---|---|---|---|---|---|---|
| trac- | T_{G} | T_{M} | T_{D} | Na | K | Ca | Mg | η | Strength |
| tion | ° C. | ° C. | ° C. | Mg/g | Mg/g | Mg/g | Mg/g | cP | g |
| Neu- | 35 | 58 | 58 | 13.70 | 46.60 | 2.90 | 5.60 | 378 | 144 |
| tral | | | | | | | | | |

TABLE 8

| | T_{G} | T_{M} | T_{D} | Na | K | Ca | Mg | η | Break |
|---|---|---|---|---|---|---|---|---|---|
| Extraction | ° C. | ° C. | ° C. | Mg/g | Mg/g | Mg/g | Mg/g | cP | Strength g |
| Traditional Kappa | 37 | 62 | 64 | 7.90 | 48.30 | 19.40 | 0.40 | 2530 | 396 |

As before, when traditional kappa carrageenan is compared to a neutrally extracted E. cottonii, traditional kappa: (1) provides gels with higher gelling and melting temperatures; (2) requires higher temperatures for dissolution; (3) has a lower content of sodium and magnesium ions; and (4) has a higher content of potassium and calcium ions.

Alkali Treatment of Wet Extract Precipitate. Wet extract was then treated with an alkali, with the results being set forth in tables 9 and 10, below.

TABLE 9

| | Treatment | | | | | | | | | | |
|---|---|---|---|---|---|---|---|---|---|---|---|
| | Temperature | Treatment | T_{G} | T_{M} | T_{D} | Na | K | Ca | Mg | η | Break |
| Extraction | ° C. | Time h | ° C. | ° C. | ° C. | Mg/g | Mg/g | Mg/g | Mg/g | cP | Strength g |
| Neutral | 25 | 0 | 36 | 57 | 59 | 13.70 | 46.60 | 2.90 | 5.60 | 378 | 144 |
| | 25 | 1 | 22 | 42 | 43 | 54.40 | 6.40 | 3.00 | 5.80 | 314 | 245 |
| | 25 | 3 | 21 | 40 | 41 | 64.30 | 6.40 | 3.00 | 6.00 | 151 | 284 |

TABLE 10

| | Treatment | | | | | | | | | | |
|---|---|---|---|---|---|---|---|---|---|---|---|
| | Temperature | Treatment | T_{G} | T_{M} | T_{D} | Na | K | Ca | Mg | η | Break |
| Extraction | ° C. | Time h | ° C. | ° C. | ° C. | Mg/g | Mg/g | Mg/g | Mg/g | cP | Strength g |
| Traditional | 25 | 0 | 37 | 62 | 64 | 7.90 | 48.30 | 19.40 | 0.40 | 2530 | 396 |
| Kappa | 25 | 1 | 21 | 39 | 41 | 40.50 | 4.20 | 19.90 | 0.40 | 142 | 245 |
| | 25 | 3 | 20 | 38 | 40 | 44.20 | 3.60 | 18.40 | 0.36 | 110 | 298 |

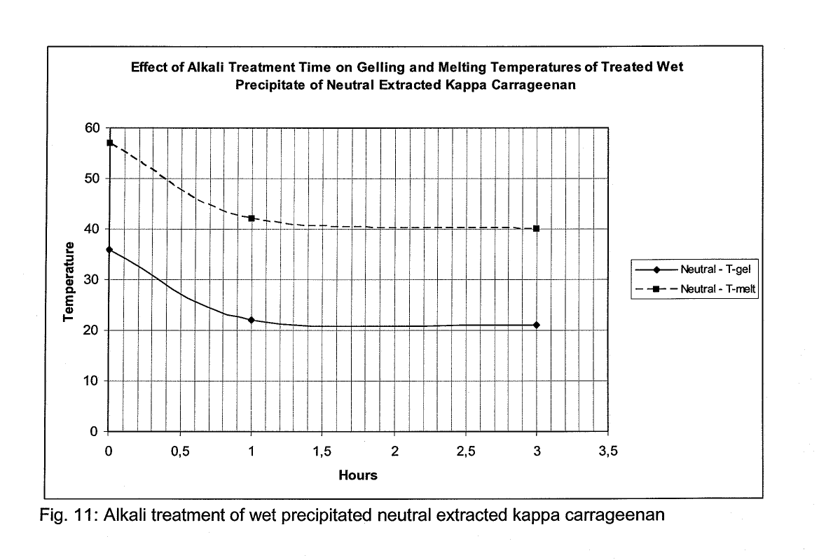

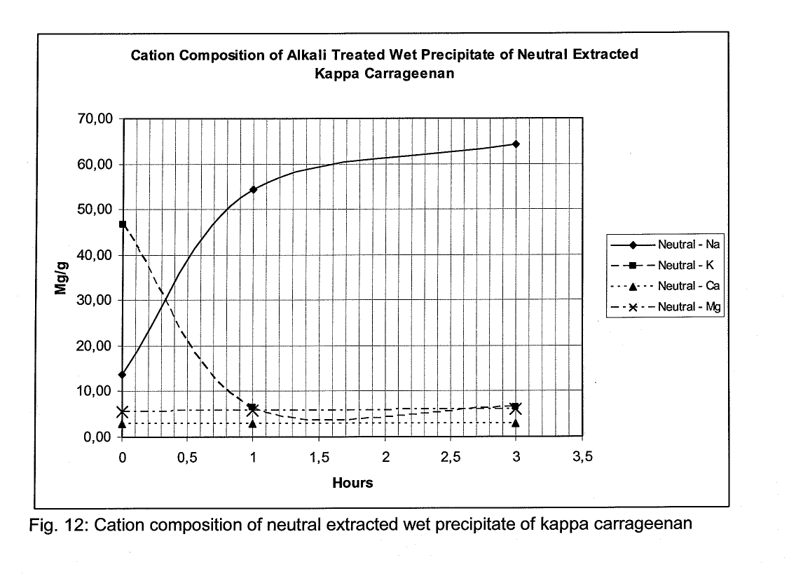

The results in table 9 are shown graphically in FIG. 11 and FIG. 12. In particular, FIG. 11 shows that gelling and melting temperatures decrease rapidly during about the first hour of alkali treatment. Gelling temperature decreases from about 36° C. to about 20° C., whereas melting temperature drops from about 57° C. to about 40° C.

FIG. 12 shows that the sodium content increases rapidly during the first hour of alkali treatment, but continues to increase with increasing alkali treatment time. The sodium content rises from about 1,3% to about 6,5%. The potassium level drops in the first our and becomes constant. It decreases from about 4,7% to about 0,5%. The calcium level stays constant at about 0,3% and the magnesium level stays constant at about 0,6%.

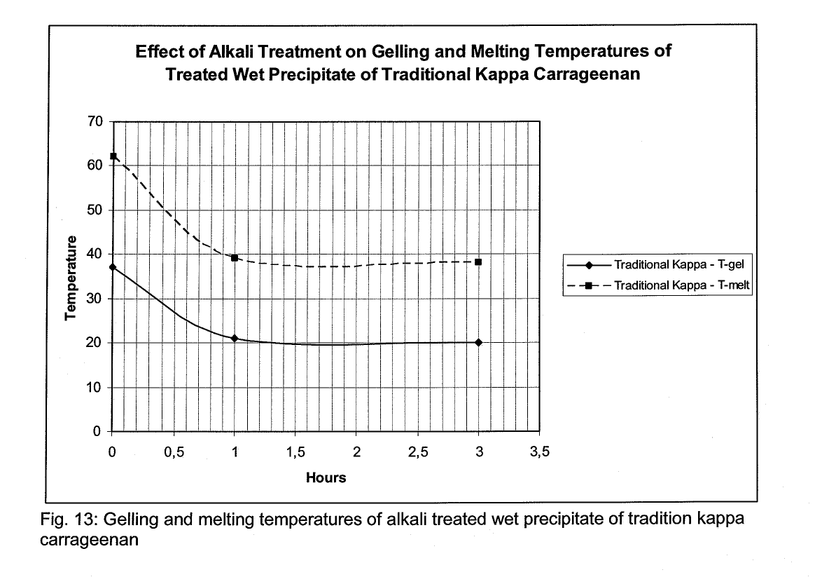

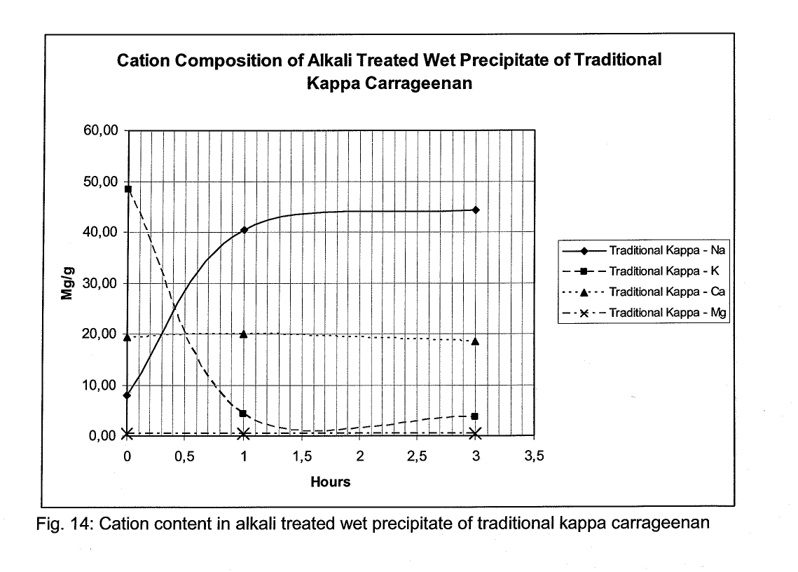

The results in table 10 are shown graphically in FIG. 13 and FIG. 14, FIG. 13 shows that gelling and melting temperatures decrease rapidly within the first hour of alkali treatment Gelling temperature drops from about 37° C. to about 20° C., whereas melting temperature drops from about 62° C. to about 38° C.

FIG. 14 show's that the sodium content increases rapidly within the first hour of alkali treatment. It goes from about 0.8% to about 4.4%. The potassium level drops rapidly during the first hour. It drops from about 5% to about 0.4%. The calcium and magnesium levels stay constant at about 2% and 0.04%, respectively.

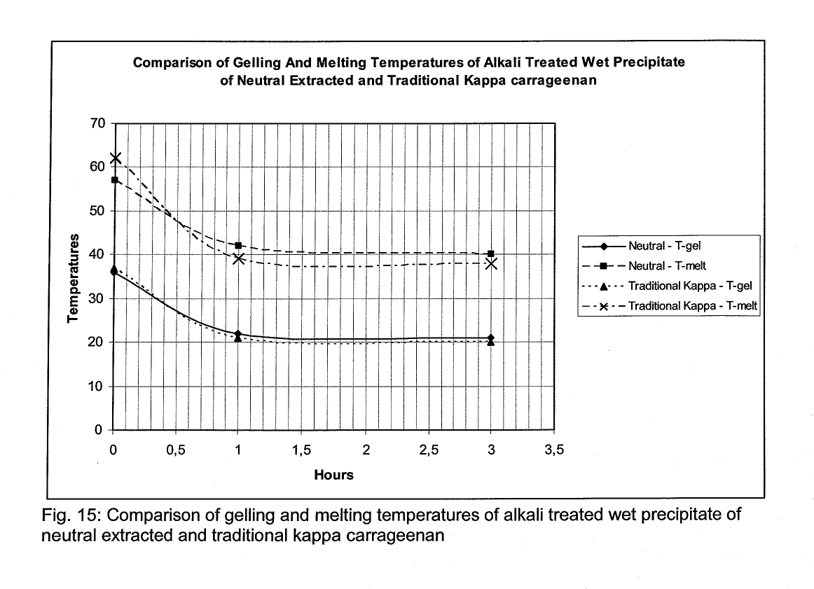

FIG. 15 compares the gelling and melting temperatures of neutral extracted kappa carrageenan and traditional kappa carrageenan. It shows that there is no difference in gelling and melting temperatures.

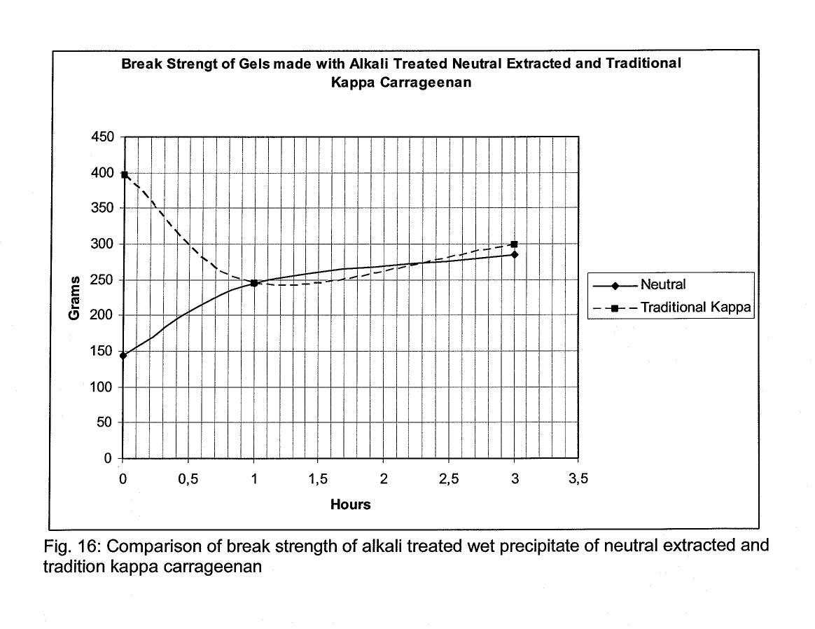

FIG. 16 compares the break strength of the two preparations. It shows that as alkali treatment commences, the break strength of traditional kappa carrageenan begins to drop whereas the break strength of neutral extracted kappa carrageenan begins to climb. After about one hour treatment, the two preparations display the same break strength. This coincides with the levels of potassium in the two preparations, which become low and practically the same after one hour treatment

Salt Treatment of Wet Extract Precipitate. Wet extract was then treated with a salt, with the results being set forth in tables 11 and 12, below.

TABLE 11

| | Treatment | | | | | | | | | | |
|---|---|---|---|---|---|---|---|---|---|---|---|
| | Temperature | Treatment | T_{G} | T_{M} | T_{D} | Na | K | Ca | Mg | η | Break |
| Extraction | ° C. | Time h | ° C. | ° C. | ° C. | Mg/g | Mg/g | Mg/g | Mg/g | cP | Strength g |
| Neutral | 25 | 0 | 35 | 58 | 58 | 13.70 | 46.60 | 2.90 | 5.60 | 378 | 144 |
| | 25 | 1 | 24 | 41 | 43 | 45.40 | 17.30 | 0.98 | 0.33 | 440 | 147 |
| | 25 | 3 | 24 | 40 | 41 | 47.80 | 14.90 | 0.94 | 0.33 | 758 | 154 |

TABLE 12

| | Treatment | | | | | | | | | | |
|---|---|---|---|---|---|---|---|---|---|---|---|
| | Temperature | Treatment | T_{G} | T_{M} | T_{D} | Na | K | Ca | Mg | η | Break |
| Extraction | ° C. | Time h | ° C. | ° C. | ° C. | Mg/g | Mg/g | Mg/g | Mg/g | cP | Strength g |
| Traditional | 25 | 0 | 37 | 62 | 64 | 7.90 | 48.30 | 19.40 | 0.40 | 2530 | 396 |
| Kappa | 25 | 1 | 21 | 38 | 41 | 41.90 | 11.00 | 4.20 | 0.23 | 141 | 400 |
| | 25 | 3 | 22 | 41 | 43 | 43.10 | 11.20 | 5.00 | 0.23 | 142 | 406 |

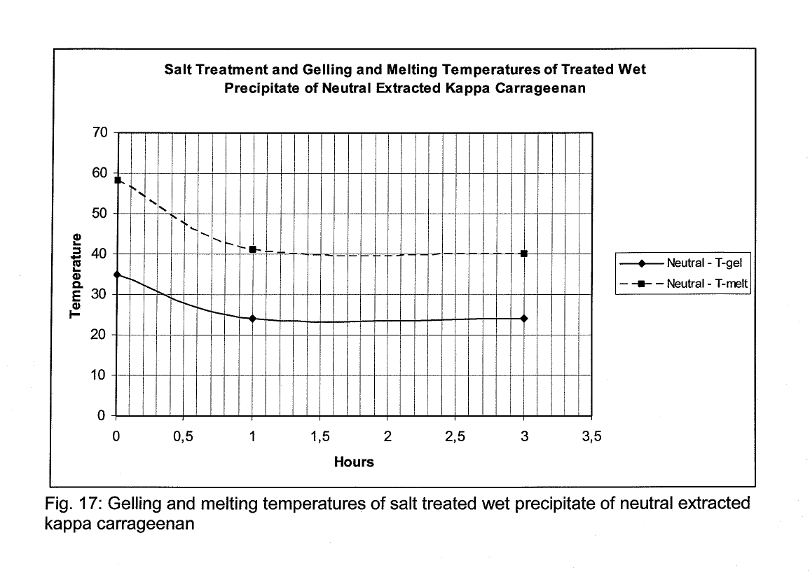

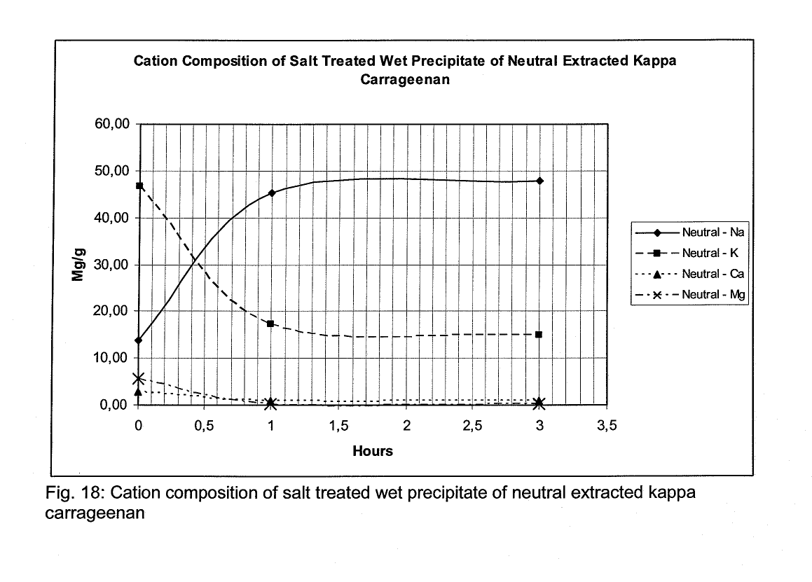

The results in table 11 are shown graphically in FIG. 17 and FIG. 18 show. FIG. 17 shows that the gelling and melting temperatures decrease to a minimum after one hour's treatment. Gelling temperature drops from about 35° C. to about 24°C., whereas melting temperature drops from about 58° C. to about 40° C. FIG. 18 shows that the sodium level reaches a maximum after about one hour's treatment, and increases from about 1.3% to about 4.6%. The potassium level reaches a minimum after about one hour's treatment, and drops from about 4.7% to about 1.5%. Likewise, calcium levels drop from about 0.3% to about 0.1% and magnesium levels from about 0.6% to about 0.03%.

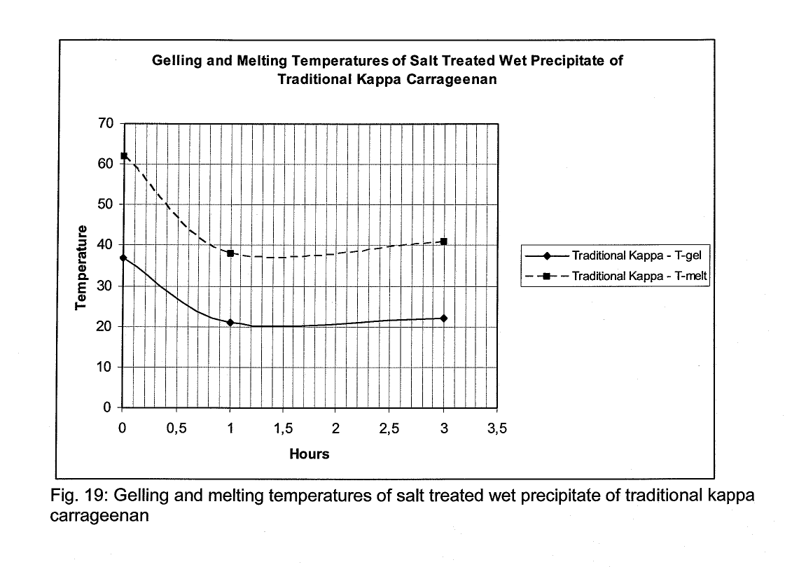

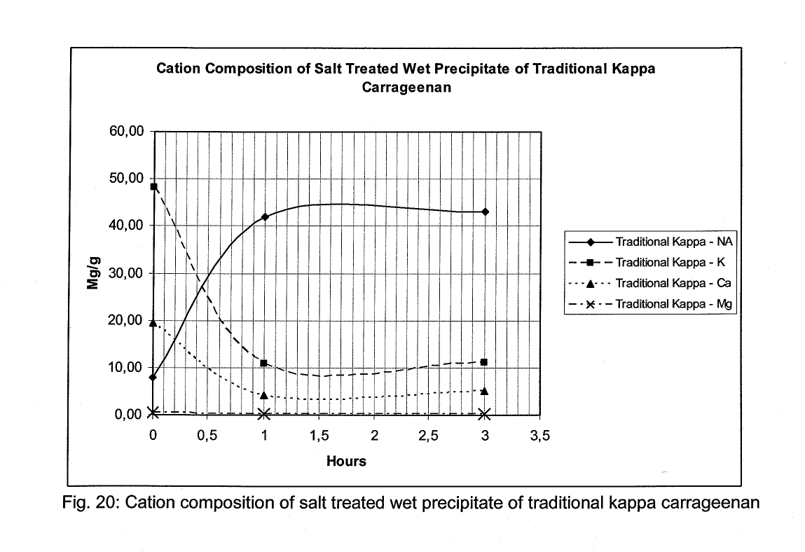

The results in table 12 are shown graphically in FIG. 19 and FIG. 20. FIG. 19 shows that gelling and melting temperatures reach a minimum after about one hour's treatment with salt. Gelling temperature drops from about 37° C. to about 20° C., whereas melting temperature drops from about 62° C. to about 38° C. FIG. 20 shows that the sodium level reaches a maximum level after about one hour's treatment and climbs from about 0.8% to about 4.5%, whereas the other cations reach a minimum after about one hour's treatment. Potassium drops from about 4.8% to about 1%, calcium from about 2% to about 0.4% and magnesium from about 0.04% to about 0.02%.

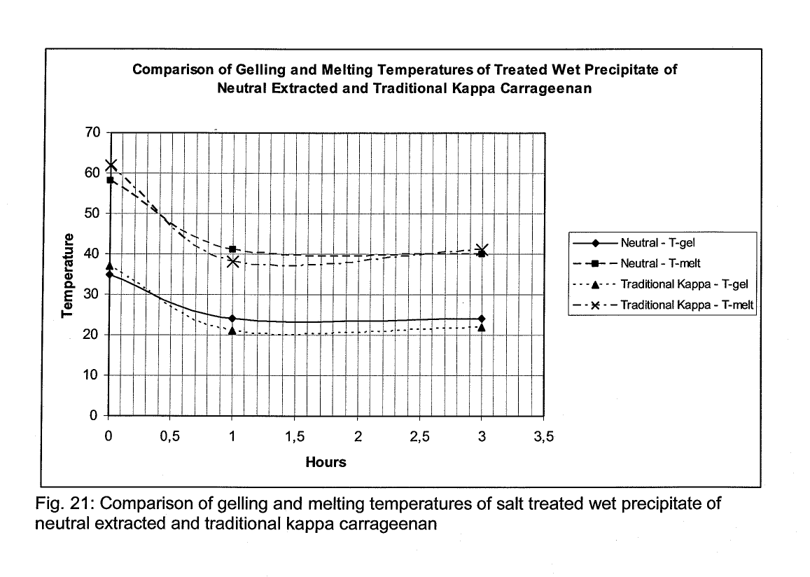

FIG. 21 compares the gelling and melting temperatures of salt treated neutral extracted and traditional kappa carrageenan. It shows that there is no difference between these temperatures.

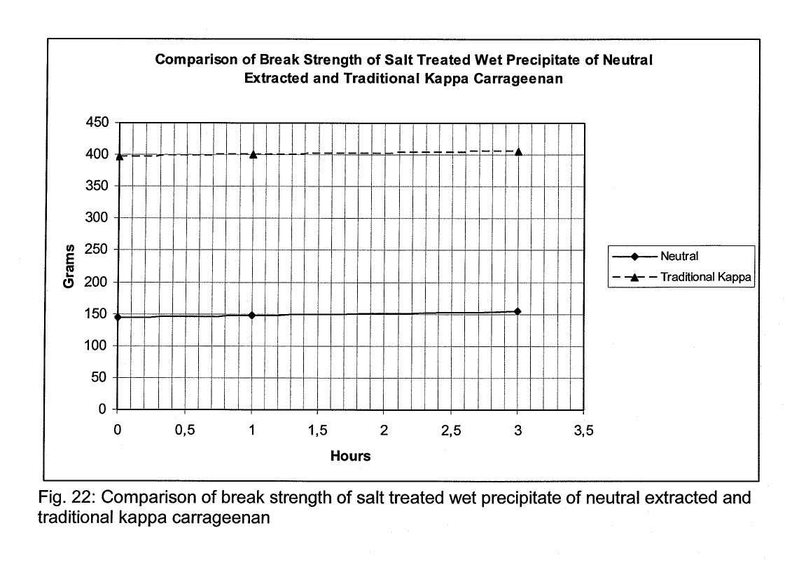

FIG. 22 compares the break strengths of the two preparations. For both preparations, the break strength stays unaffected by the treatment time. However, the traditional kappa carrageenan provides substantially higher break strength than the neutral extracted kappa carrageenan.

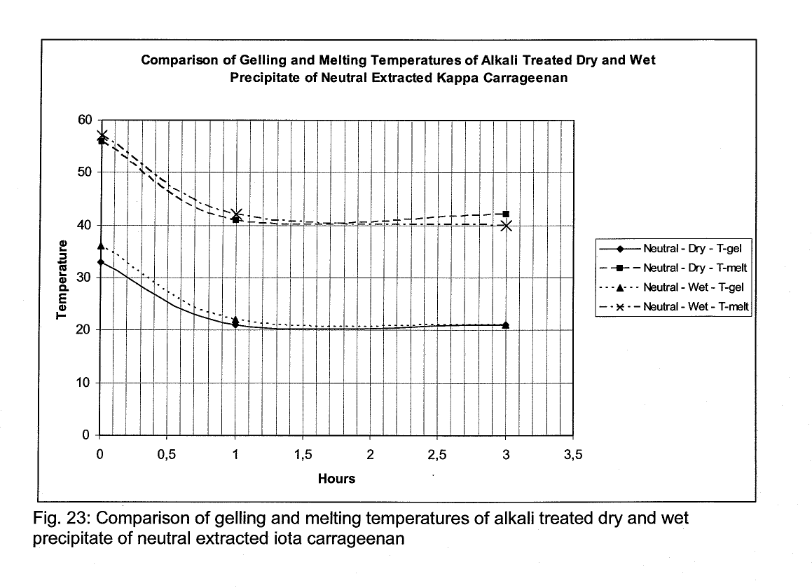

FIG. 23 shows a comparison of gelling and melting temperatures of alkali treated dry and wet precipitate of neutral extracted iota carrageenan. It shows that there is very little difference in gelling and melting temperatures whether the treated precipitate id dry or wet.

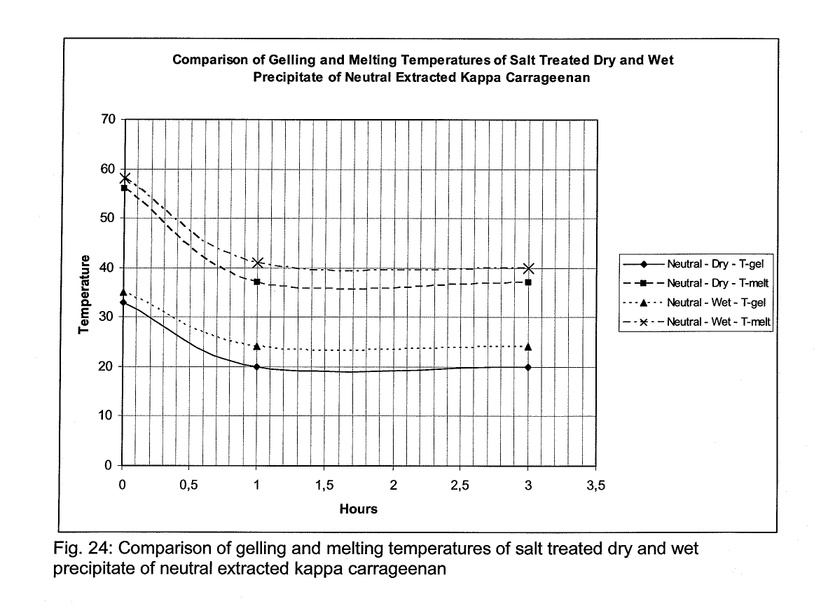

FIG. 24 shows the same plot as FIG. 23, but with salt treated precipitate. It shows that there is less than 3° C. difference in gelling and melting temperatures depending on whether dry or wet precipitate is treated.

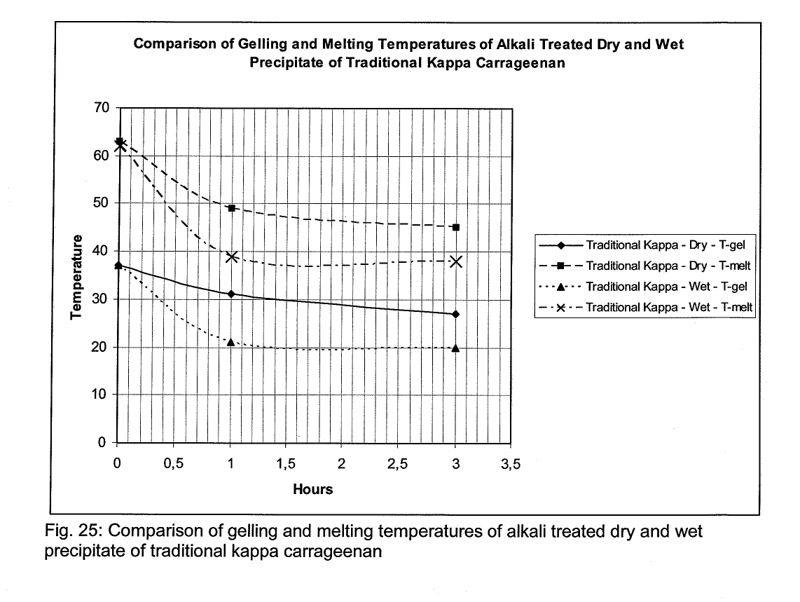

FIG. 25 shows the same plot as FIG. 23, but for traditional kappa carrageenan. It shows that for traditional kappa carrageenan, the state of the precipitate plays a role. Gelling temperature can be adjusted within the range from about 20° C. to about 37° C. and melting temperature within the range from about 38° C. to about 63° C.

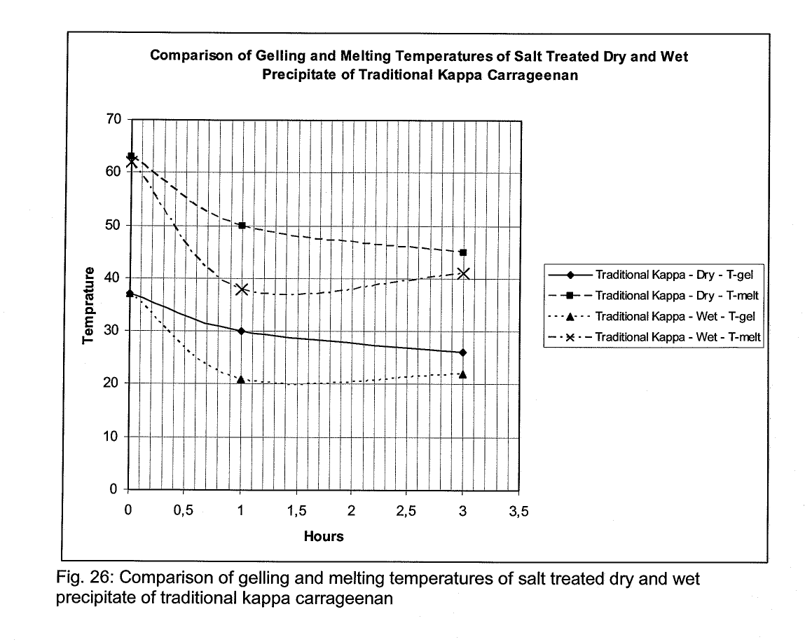

FIG. 26 shows the same plot as FIG. 25 but with salt treatment. It shows again that for traditional kappa carrageenan, the state of the precipitate matters. Gelling temperatures can be adjusted in the range from about 21° C. to about 37° C. and melting temperatures from about 38° C. to about 63°C.

Salt Treatment of Wet Extract Precipitate with lower alcohol concentrations. A similar procedure to that used above to produce the data shown in tables 11 and 12 was used except that alcohol concentration was kept at a lower level.

Specifically, the extract was traditional kappa carrageenan, treated at 25° C., however, instead of using 120 ml ethanol and 80 ml demineralized water, the treatment used 20 ml ethanol and 80 ml demineralized water.

TABLE 13

| | Treatment | | | | | | | | | | |
|---|---|---|---|---|---|---|---|---|---|---|---|
| | Temperature | Treatment | T_{G} | T_{M} | T_{D} | Na | K | Ca | Mg | η | Break |
| Extraction | ° C. | Time h | ° C. | ° C. | ° C. | Mg/g | Mg/g | Mg/g | Mg/g | cP | Strength g |
| Traditional | 25 | 1 | 20 | 37 | 41 | 69.90 | 12.70 | 0.80 | 0.30 | 190 | 429 |
| Kappa | | | | | | | | | | | |

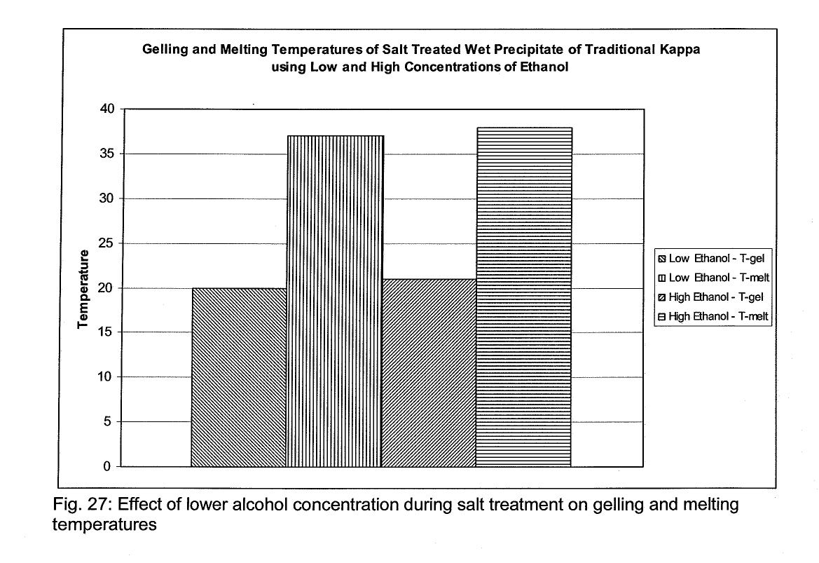

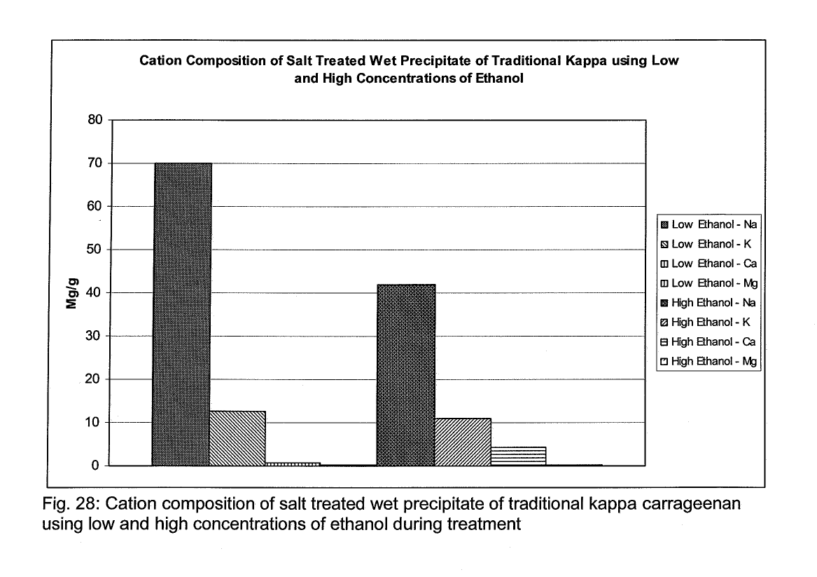

The results in Table 13 are shown graphically in FIG. 27 and FIG. 28, along with the corresponding values from table 12.

FIG. 27 shows that there is a slight decrease in gelling and melting temperatures when salt treatment takes place with less alcohol.

FIG. 28 shows that compared to high alcohol concentration during treatment, low alcohol during treatment provides substantially higher content of sodium and slightly higher content of potassium. However, the calcium content is substantially lower.

Without being bound of theory, it is believed that the higher concentration of water during salt treatment provides for an improved diffusion of ions, which particularly increases the sodium level and reduces the calcium level. The potassium, however, requires more time, possibly because potassium is tighter bound inside the carrageenan structure.

It will be appreciated by those skilled in the art that changes could be made to the embodiments described above without departing from the broad inventive concept thereof. It is understood therefore that this invention is not limited to the particular embodiments disclosed, but it is intended to cover modifications within the spirit and scope of the present invention as defined by the appended claims.

1. A process for treating precipitated carrageenan, comprising the steps of (a) treating the precipitated carrageenan with an aqueous treatment solution containing an alkali or a salt, (b) washing the treated precipitated carrageenan in water, and (c) drying the washed precipitated carrageenan.

2. The process according to claim 1, wherein before the treating step, the precipitated carrageenan is obtained by extraction of red seaweed.

3. The process according to claim 1, wherein the precipitated carrageenan is kappa carrageenan.

4. The process according to claim 1, wherein the precipitate is wet.

5. The process according to claim 1, wherein the precipitate is dry.

6. The process according to claim 5, wherein the precipitate is a powder.

7. The process according to claim 1, wherein the aqueous treatment solution contains an aqueous salt in a concentration of about 3 to about 30 wt%, preferably about 10-25 wt%, and most preferably about 15 to about 20 wt%.

8. The process according to claim 1, wherein the aqueous treatment solution contains an aqueous alkali in a concentration of about 3 to about 30 wt%, preferably about 10-25 wt%, and most preferably about 15 to about 20 wt%.

9. The process according to claim 1, wherein the treatment step is conducted at a treatment temperature of 0-70° C., preferably 5-50° C. and most preferably 5-25° C., and wherein the treatment time is in the range 1 minute to 24 hours, preferably 1 minute to 5 hours, and most preferably 1 minute to 80 minutes.

10. The process according to claim 1, wherein the (b) washing step occurs with slow agitation, and further comprises 1-4, preferably 1-2, washings, with each washing lasting in the range of 10-30 minutes, preferably 15 minutes per wash, and wherein the temperature during washing is in the range of 0-25° C., preferably 0-5° C.

11. The process according to claim 1 wherein the treatment is performed batch wise or in counter current process.

12. The process according to claim 8, wherein the aqueous alkali is an alkali of sodium.

13. The process according to claim 8, wherein the aqueous alkali is selected from the group comprising sodium hydroxide, sodium carbonate, and sodium bicarbonate.

14. The process according to claim 1, wherein the aqueous treatment solution further comprises alcohol in a concentration of about 20 vol% to about 80 vol%, preferably about 20 vol% to about 60 vol%, most preferably about 20 vol% to about 50 vol%.

15. The process according to claim 14 where the alcohol is selected from the group comprising methanol, ethanol, isopropyl alcohol.

16. The process according to claim 1 in which the aqueous treatment solution contains salt.

17. The process according to claim 1 in which the aqueous treatment solution contains a salt selected from the comprising sodium chloride, sodium sulphate, sodium phosphate, sodium tripolyphosphate and sodium hexametaphosphate.

18. A carrageenan composition prepared according to the process of claim 8, the carrageenan composition comprising: sodium in the range of 4.050-7.310%, preferably 4.420-7.310% and most preferably 5.440-7.310%; potassium in the range of 0.320%-4.560%, preferably 0.320-0.910% and most preferably 0.320-0.640%; calcium in the range of 0.300-1.990%, preferably 0.300-1.790% and most preferably 0.300-1.620%; and magnesium in the range of 0.012-0.630%, preferably 0.012-0.600% and most preferably 0.012-0.580%; wherein the carrageenan's gelling temperature is in the range 20-37° C., preferably 20-31° C. and most preferably 20-22° C.; and the carrageenan's melting temperatures in the range 38° C.-63° C., preferably 38° C.-49° C. and most preferably 38° C. -40° C.

19. A food producing comprising the carrageenan of claim 18.

20. A food product comprising the carrageenan of claim 18, wherein the food product is selected from the group comprising processed meat, poultry, and a fish product.

21. A food producing comprising the carrageenan of claim 18, wherein the food products is a water-in-oil emulsion.

22. A household product comprising the carrageenan of claim 18.

23. A personal care product comprising the carrageenan of claim 18, wherein the personal care product is a water-in-oil emulsion comprising 20-80% oil, and where said emulsion inverts at any temperature in the range 37-50° C., preferably 37-41° C. to ensure inversion on the skin surface.

24. A toothpaste comprising the carrageenan of claim 18.

25. A pharmaceutical product comprising the carrageenan of claim 18.

26. A pharmaceutical product comprising the carrageenan of claim 18, wherein the pharmaceutical is in the form of a soft capsule.

27. A pharmaceutical product comprising the carrageenan of claim 18, wherein the pharmaceutical is in the form of an encapsulated heat sensitive drug.

28. A pharmaceutical product comprising the carrageenan of claim 18, wherein the pharmaceutical product is an encapsulated drug, which must be released at temperatures in the range 37-50° C., preferably 37-41° C.

29. A method for flavor encapsulation comprising the carrageenan of claim 18, wherein the flavor is to be released at temperatures in the range 37-50° C.

30. A carrageenan composition prepared according to the process of claim 7, the carrageenan composition comprising: sodium in the range 3.730-6.990%, preferably 4.190-6.990% and most preferably 4.310-6.990%; potassium in the range of 0.840-4.560%, preferably 0.840-1.730% and most preferably 0.840-1.490%; calcium in the range of 0.080-1.750%, preferably 0.080-0.500% and most preferably 0.080-0.420%; and magnesium in the range of 0.005-0.610%, preferably 0.005-0.030% and most preferably 0.005-0.023%; and wherein the carrageenan composition has gelling temperatures in the range 19-37° C., preferably 19-24° C. and most preferably 19-22° C.; and the carrageenan composition has melting temperatures in the range 37-63° C., preferably 37-42° C. and most preferably 37-40° C.

31. A food producing comprising the carrageenan of claim 30.

32. A food product comprising the carrageenan of claim 30, wherein the food product is selected from the group comprising processed meat, poultry, and a fish product.

33. A food producing comprising the carrageenan of claim 30, wherein the food products is a water-in-oil emulsion.

34. A household product comprising the carrageenan of claim 30.

35. A personal care product comprising the carrageenan of claim 30, wherein the personal care product is a water-in-oil emulsion comprising 20-80% oil, and where said emulsion inverts at any temperature in the range 15-45° C., preferably 30-35° C. to ensure inversion on the skin surface.

36. A toothpaste comprising the carrageenan of claim 30.

37. A pharmaceutical product comprising the carrageenan of claim 30.

38. A pharmaceutical product comprising the carrageenan of claim 30, wherein the pharmaceutical is in the form of a soft capsule.

39. A pharmaceutical product comprising the carrageenan of claim 30, wherein the pharmaceutical is in the form of an encapsulated heat sensitive drug.

40. A pharmaceutical product comprising the carrageenan of claim 30, wherein the pharmaceutical product is an encapsulated drug, which must be released at temperatures in the range 37-50° C., preferably 37-41° C.

41. A method for flavor encapsulation comprising the carrageenan of claim 30, wherein the flavor is to be released at temperatures in the range 37-50° C.
